(19) United States
(12) Patent Application Publication
(10) Pub. No.: US 2015/0140134 A1
(43) Pub. Date: May 21, 2015

(54) Natural cream pain reliever

(71) Applicant: Harry McPherson, Las Vegas, NV (US)

(72) Inventor: Harry McPherson, Las Vegas, NV (US)

(21) Appl. No.: 13/998,635

(22) Filed: Nov. 20, 2013

(51) Int. Cl. A61K 36/889

(52) U.S. Cl. CPC A61K 36/889
USPC 424/727

(57) ABSTRACT

The Natural Cream Pain Reliever is a 100% natural formula with coconut as the main component and a manufacturing process using refrigeration to extract the purist and most concentrated portion of the ingredients.

There is no federally sponsored or research or development associated with this application.

There is no joint research agreement associated with this application.

### BACKGROUND OF THE INVENTION

(1) Field of the Invention

The Natural Cream Pain Reliever is a 100% natural product and invention that uses a specific formula and manufacturing process compared to others on the market.

(2) Description of the Art Including Information Disclosed Under 37 CFR 1.97 & 1.98

The art described in this application is unique to anything we have found to this point. U.S. Pat. No. 8,425,954 was used to establish the proper claim format.

### BRIEF SUMMARY OF THE INVENTION

The Natural Cream Pain Reliever is a 100% natural product using coconut as the main ingredient and is manufactured in a very specific process to achieve a concentrated paste that rubs into the skin over the pain affected area.

### BRIEF DESCRIPTION OF THE SEVERAL VIEWS OF THE DRAWINGS

There are no drawings within this specification and application.

### DETAILED DESCRIPTION OF THE PREFERRED EMBODIMENT

There are no drawings within this specification and application.

### DETAILED DESCRIPTION OF THE INVENTION

Natural pain relievers are not new and the coconut has been attributed to a natural pain reliever for a very long time into ancient history. The healing properties of coconut and coconut oil primarily have been recorded for oral use and for topical use on the skin around the pain affected area. The coconut is basically pressed into an oil form that is the purist form of that extraction process. However it has also been noted that coconut oil as with any natural oil will become rancid in a short period of time unless it is frozen or kept refrigerated. Even when refrigerated it seems to lose some of its healing qualities because the oil is very sensitive to cold.

Because of the natural fatty acids called medium chain fatty acids it can be used as an anti-inflammatory, a pain reliever, and as a muscle and joint lubricant and relaxant as stated in numerous natural wellness articles and in numerous medical studies. One of the biggest problems is the storage and expiration issues of the coconut oil itself. The oil wants to break down very quickly if not refrigerated or it breaks down when refrigerated as well. This is because the oil is a pure extract and does have the qualities of having the original coconut fiber to help it last longer.

The formula and process by which this new product is manufactured is what makes this separate from other coconut oils that are pressed into a concentrate. With this new process it is not pressed into an oil but is formed into a concentrated state using the fiber of the coconut. It is best to use older coconuts because there is more coconut meat to work with and higher concentrations of the oil in the fiber of the coconut meat. The first step in the process is to pull or chop the meat of the coconut from the shell and mash it up as best as possible. Then alkaline water is added to the coconut meat in a blender with a 55% meat and a 45% alkaline water. Blend the mixture for about 30 to 60 seconds until it is soft and flakey but not liquid. It has to be in a solid state about the same consistency of a horseradish mixture found in grocery stores such as a Morehouse brand.

From there it is strained into a regular type of strainer and then a second strainer into a bowl where you then place that container into a refrigerator overnight or for 24 hours is best.

Over the next 24 hours the water in the mixture will separate from the meat and you will see a more solid paste that is concentrated without all of the water. This is much better than a pressed oil because it has all the properties needed for the healing process without it being in an oil state. Once you have placed the concentrated paste or cream at this point into another container place it back into the refrigerator and then use it as desired.

The cream can be frozen for transportation and it does not affect the cream in any way. If the cream is allowed to warm up and turn to liquid the cream is no longer affective in the pain relieving process. Always keep the cream refrigerated. You apply the cream directly onto the skin around the pain affected area. Use a generous amount and rub or massage into the area affected and continue to rub it in until it has disappeared. Unlike an oil it will not leave an oily residue and you can watch it being absorbed into the skin. You can use the cream once a day for a few days in a row or once a week depending on the affected area and the degree of pain.

This formula and process has been used by many people and most people have seen remarkable results such as joint pain that they no longer need shots or medication for the pain. The manufacturing process and the refrigeration process are what makes this product different from anything else in the market and is what makes it work as well as it does.

What is claimed is:

1. A method of manufacturing a natural cream for pain relief, the method comprising the steps of:
(a) scraping the white meat from a mature coconut and mixing it with alkaline water;
(b) mixing the white meat and the alkaline water at a 55% meat and 45% alkaline water ratio;
(c) placing the mix into a blender for 30 to 60 seconds;
(d) straining the blended mix 2 times into a separate container;
(e) placing the container into the refrigerator for 24 hours.

2. The method of claim 1 wherein after 24 hours removing the white paste or cream from the container now separated from the water and placing into another container and placing into the refrigerator for use.
